(19) United States
(12) Patent Application Publication
(10) Pub. No.: US 2024/0136023 A1
(43) Pub. Date: Apr. 25, 2024

(54) DATA PROCESSING METHOD AND APPARATUS, DEVICE, AND STORAGE MEDIUM

(71) Applicant: Tencent Technology (Shenzhen) Company Limited, Shenzhen (CN)

(72) Inventors: Zihan WU, Shenzhen (CN); Jianhua Yao, Shenzhen (CN)

(21) Appl. No.: 18/396,522

(22) Filed: Dec. 26, 2023

Related U.S. Application Data
(63) Continuation of application No. PCT/CN23/95787, filed on May 23, 2023.

(30) Foreign Application Priority Data
Jul. 22, 2022 (CN) 202210871878.7

(51) Int. Cl. G16B 45/00; G06T 11/20; G16B 25/10; G16B 40/20

(52) U.S. Cl. CPC G16B 45/00; G06T 11/206; G16B 25/10; G16B 40/20

(57) ABSTRACT

This application provides a data processing method performed by an electronic device. The method includes: obtaining target data, and parsing the target data to obtain spatial location information and first feature information of each object in N objects; converting the target data into a first image based on the spatial location information and the first feature information of each object; extracting second feature information of each object in the target data from the first image; and performing preset processing on the second feature information of each object in the target data, to obtain a processing result of the target data. The entire data processing process is simple, occupies less computing resources, and has high data processing efficiency.

### RELATED APPLICATION

This application is a continuation application of PCT Patent Application No. PCT/CN2023/095787, entitled “DATA PROCESSING METHOD AND APPARATUS, DEVICE, AND STORAGE MEDIUM” filed on May 23, 2023, which claims priority to Chinese Patent Application No. 202210871878.7, filed with the China National Intellectual Property Administration on Jul. 22, 2022 and entitled “DATA PROCESSING METHOD AND APPARATUS, DEVICE, AND STORAGE MEDIUM”, all of which is incorporated herein by reference in its entirety.

### FIELD OF THE TECHNOLOGY

Embodiments of this application relate to the field of computer technologies, and in particular, to a data processing method and apparatus, a device, and a storage medium.

### BACKGROUND OF THE DISCLOSURE

For some data, spatial location information of the data needs to be considered during data processing, for example, spatial transcriptomic data. The spatial transcriptomic data includes gene information and spatial location information of a cell. In a clustering scenario of the spatial transcriptomic data, to achieve clustering accuracy, clustering needs to be performed with reference to the gene information and the spatial location information of the cell.

However, when being configured for performing clustering on the spatial transcriptomic data, a current data processing technology, for example, a clustering technology of the spatial transcriptomic data occupies a relatively large quantity of computing resources and has low efficiency.

### SUMMARY

This application provides a data processing method and apparatus, a device, and a storage medium, which can reduce an occupied amount of computing resources and improve data processing efficiency.

According to a first aspect, an embodiment of this application provides a data processing method, including:
obtaining target data, and parsing the target data to obtain spatial location information and first feature information of each object in the target data;
converting the target data into a first image based on the spatial location information and the first feature information of each object;
extracting second feature information of each object in the target data from the first image; and
performing preset processing on the second feature information of each object in the target data.

According to a second aspect, an embodiment of this application provides a data processing apparatus, including:
an obtaining unit, configured to obtain target data, and parse the target data to obtain spatial location information and first feature information of each object in the target data;
a conversion unit, configured to convert the target data into a first image based on the spatial location information and the first feature information of each object;
an extraction unit, configured to extract second feature information of each object in the target datafrom the first image; and
a processing unit, configured to perform preset processing on the second feature information of each object in the target data.

According to a third aspect, an embodiment of this application provides an electronic device, including a processor and a memory. The memory is configured to store a computer program, and the processor is configured to invoke and run the computer program stored in the memory, to perform the method in the first aspect.

According to a fourth aspect, an embodiment of this application provides a chip, configured to implement the method according to any aspect or each implementation in the first aspect or the second aspect. Specifically, the chip includes: a processor, configured to invoke a computer program from a memory and run the computer program, to enable a device equipped with the chip to perform the method in the first aspect.

According to a fifth aspect, an embodiment of this application provides a non-transitory computer-readable storage medium, configured to store a computer program, the computer program enabling an electronic device to perform the method in the first aspect.

According to a sixth aspect, an embodiment of this application provides a computer program product, including computer program instructions, the computer program instructions enabling a computer to perform the method in the first aspect.

According to a seventh aspect, an embodiment of this application provides a computer program, when being run on a computer, the computer program enabling the computer to perform the method in the first aspect.

Based on the foregoing, in the embodiments of this application, target data is obtained, and the target data is parsed to obtain spatial location information and first feature information of each object in N objects. The target data is converted into a first image based on the spatial location information and the first feature information of each object. Second feature information of each object in the N objects is extracted from the first image, the second feature information combining the spatial location information and the first feature information. In this way, preset processing (for example, object clustering or object annotation) is performed by using the second feature information of the N objects, to obtain an accurate processing result. That is, in the embodiments of this application, the target data is converted into the first image, the first image including the spatial location information and the first feature information of each object in the N objects, then feature extraction is performed on the first image, to obtain the second feature information of each object in the N objects, to encode the spatial location information into a feature, and preset processing such as clustering is directly performed on the second feature information including the spatial location information. The entire data processing process is simple, occupies less computing resources, and has high data processing efficiency. In addition, in the embodiments of this application, when the target data is processed, the target data is converted into the first image, and feature extraction is performed on the first image. When feature extraction is performed on the first image, only the second feature information of each object in the N objects is extracted. If the first image includes a pixel whose pixel value is zero, feature extraction is not performed on the pixel whose pixel value is zero in the first image, to further save the computing resources and improve the data processing efficiency.

### BRIEF DESCRIPTION OF THE DRAWINGS

To describe the technical solutions in the embodiments of this application more clearly, the following briefly introduces the accompanying drawings required for describing the embodiments. Apparently, the accompanying drawings in the following description show only some embodiments of this application, and a person of ordinary skill in the art may still derive other accompanying drawings from these accompanying drawings without creative efforts.

FIG. 1 is a schematic diagram of an application scenario according to an embodiment of this application.

FIG. 2 is a schematic diagram of clustering of spatial transcriptomic data.

FIG. 3 is a schematic diagram of a principle of scan equalization digital radiography (SEDR).

FIG. 4 is a schematic flowchart of a data processing method according to an embodiment of this application.

FIG. 5 is a schematic diagram of a first image and a first feature map according to an embodiment of this application.

FIG. 6A to FIG. 6D are schematic diagrams of an input image, a result of ordinary convolution, a result of ordinary sparse convolution, and a result of submanifold sparse convolution.

FIG. 7 is a schematic diagram of a data processing process according to an embodiment of this application.

FIG. 8 is a schematic diagram of another data processing process according to an embodiment of this application.

FIG. 9 is a schematic diagram of training a submanifold sparse convolution-based autoencoder according to an embodiment of this application.

FIG. 10 is a schematic diagram of a processing process of spatial transcriptomic data according to an embodiment of this application.

FIG. 11 is a schematic diagram of an annotation result on one sample in rat brain primary motor cortex data in multiplexed error-robust fluorescence in situ hybridization (MERFISH) according to this application.

FIG. 12 is a schematic block diagram of a data processing apparatus according to an embodiment of this application.

FIG. 13 is a schematic block diagram of an electronic device according to an embodiment of this application.

### DESCRIPTION OF EMBODIMENTS

The technical solutions in the embodiments of this application are described below with reference to the accompanying drawings in the embodiments of this application.

It is to be understood that in the embodiments of this application, “B corresponding to A” indicates that B is associated with A. In an implementation, B may be determined according to A. However, it is to further be understood that, determining B according to A does not mean that B is determined according to A only, but B may also be determined according to A and/or other information.

In addition, in the descriptions of this application, unless otherwise specified, “a plurality of” means two or more than two.

In addition, to clearly describe the technical solutions in the embodiments of this application, terms such as “first” and “second” are used in embodiments of this application to distinguish between same items or similar items that provide basically same functions or purposes. A person skilled in the art may understand that the terms such as “first” and “second” do not limit a quantity or an execution sequence, and the terms such as “first” and “second” do not indicate a definite difference.

For ease of understanding of the embodiments of this application, related concepts in the embodiments of this application are described below first.

Artificial intelligence (AI) is a theory, method, technology, and application system that uses a digital computer or a machine controlled by the digital computer to simulate, extend, and expand human intelligence, perceive an environment, obtain knowledge, and use knowledge to obtain an optimal result. In other words, AI is a comprehensive technology in computer science and attempts to understand the essence of intelligence and produce a new intelligent machine that can react in a manner similar to human intelligence. AI is to study the design principles and implementation methods of various intelligent machines, to enable the machines to have the functions of perception, reasoning, and decision-making.

The AI technology is a comprehensive discipline, and relates to a wide range of fields including both hardware-level technologies and software-level technologies. The basic AI technologies generally include technologies such as a sensor, a dedicated AI chip, cloud computing, distributed storage, a big data processing technology, an operating/interaction system, and electromechanical integration. AI software technologies mainly include several major directions such as a computer vision (CV) technology, a speech processing technology, a natural language processing technology, and machine learning/deep learning.

Machine learning (ML) is a multi-field interdisciplinary subject involving the probability theory, statistics, the approximation theory, convex analysis, the algorithm complexity theory, and the like, ML specializes in studying how a computer simulates or implements a human learning behavior to obtain new knowledge or skills, and reorganize an existing knowledge structure to keep improving its performance. The ML, as the core of AI, is a basic way to make the computer intelligent, and is applicable to various fields of AI. ML and deep learning generally include technologies such as an artificial neural network, a belief network, reinforcement learning, transfer learning, inductive learning, and learning from demonstrations.

With the research and progress of the AI technology, the AI technology is studied and applied in a plurality of fields such as a common smart home, a smart wearable device, a virtual assistant, a smart speaker, smart marketing, unmanned driving, automatic driving, an unmanned aerial vehicle, a robot, smart medical care, and smart customer service. It is believed that with the development of technologies, the AI technology will be applied to more fields, and play an increasingly important role.

In the embodiments of this application, data processing is implemented by means of the AI technology, for example, processing such as clustering, type annotation, and downstream analysis on target data is implemented.

First, an application scenario of this embodiment of this application is described.

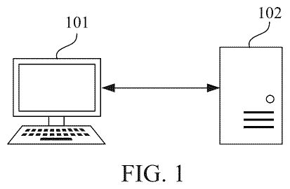

FIG. 1 is a schematic diagram of an application scenario according to an embodiment of this application, including a terminal device 101 and a server 102.

The terminal device 101 may include, but not limited to, a personal computer (PC), a PDA (a tablet computer), a mobile phone, a wearable intelligent device, a medical device, and the like. The device is generally provided with a display apparatus. The display apparatus may alternatively be a display, a display screen, a touch screen, or the like. The touch screen may alternatively be a touch screen, a touch panel, or the like. The display apparatus may be configured to display a processing result and the like.

There may be one or more servers 102. When there are a plurality of servers 102, at least two servers are configured to provide different services, and/or at least two servers are configured to provide a same service, for example, provide the same service in a load balancing manner This is not limited in this embodiment of this application. The server 102 may be an independent physical server, or may be a server cluster or a distributed system formed by a plurality of physical servers, or may be a cloud server that provides a basic cloud computing service such as a cloud service, a cloud database, cloud computing, a cloud function, cloud storage, a network service, cloud communication, a middleware service, a domain name service, a security service, a content delivery network (CDN), big data, and an artificial intelligence platform. The server 102 may alternatively be a node of a blockchain.

The terminal device 101 and the server 102 may be directly or indirectly connected in a wired or wireless communication manner This is not limited in this embodiment of this application.

In some embodiments, the server 102 in this embodiment of this application may train a feature extraction model (for example, a submanifold sparse convolution-based autoencoder) in the embodiments of this application and store the trained feature extraction model.

In some embodiments, the data processing method in the embodiments of this application may be completed by the terminal device 101. For example, the terminal device 101 obtains target data and obtains a processing result of the target data according to the method provided in the embodiments of this application. In an example, the terminal device 101 loads the trained feature extraction model in the server 102, performs feature extraction on a first image by using the feature extraction model, to obtain second feature information of each object in N objects, and performs preset processing on the second feature information of each object in the N objects, to obtain the processing result of the target data.

In some embodiments, the data processing method in the embodiments of this application may be completed by the server 102. For example, the terminal device 101 sends target data to the server 102, and the server 102 processes the target data according to the method provided in the embodiments of this application, to obtain a processing result of the target data. In an example, the server 102 loads the trained feature extraction model stored in the server 102, performs feature extraction on a first image corresponding to the target data, to obtain second feature information of each object in N objects, and performs preset processing on the second feature information of each object in the N objects, to obtain the processing result of the target data. In some embodiments, the server 102 may further send the processing result to the terminal device 101 for display.

In some embodiments, the data processing method in the embodiments of this application may be jointly completed by the terminal device 101 and the server 102. For example, the server 102 performs an operation related to a network model, and the terminal device 101 performs another operation than the operation related to the network model. For example, the terminal device 101 obtains target data, converts the target data into a first image, and sends the first image to the server 102. The server 102 performs feature extraction on the first image, for example, performs feature extraction on the first image by using the trained feature extraction model, to obtain second feature information of each object in N objects. Then, the server performs preset processing on the second feature information of each object in the N objects, to obtain a processing result of the target data, and finally sends the processing result to the terminal device 101 for display.

The application scenario in this embodiment of this application includes, but not limited to, that shown in FIG. 1.

The data processing method provided in this embodiment of this application is applicable to any data processing application that needs to combine spatial location information.

In some embodiments, this embodiment of this application is applicable to a clustering scenario of spatial transcriptomic data.

A spatial transcriptome sequencing technology is a new technology in recent years, and the key of the spatial transcriptome sequencing technology lies in that in addition to gene expression of a cell, spatial location information of the cell that is not available for single cell transcriptomic data can also be obtained.

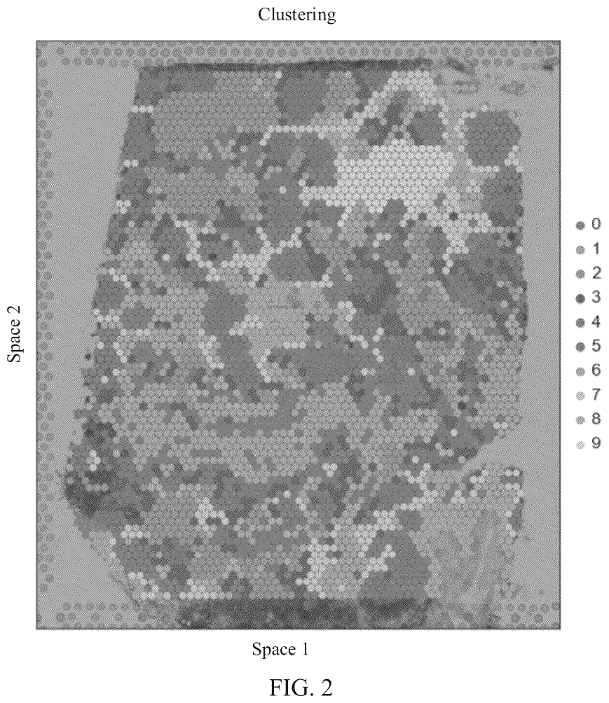

A main objective of clustering analysis of the spatial transcriptomic data is to divide a group of cells into a plurality of groups according to gene expression of each cell. Currently, a general clustering analysis process is mainly divided into two steps: First, feature extraction is performed, and then clustering is performed on an extracted feature by using a clustering algorithm. The clustering algorithm may be a general clustering algorithm such as a K-means clustering algorithm (K-means) or a K-nearest neighbor (KNN) algorithm, or a clustering algorithm such as Louvain or Leiden based on community detection that is more suitable for cell clustering. For example, FIG. 2 is a schematic diagram of clustering of spatial transcriptomic data.

A core of clustering of the spatial transcriptomic data is a feature extraction process. A group of cell data generally includes tens of thousands of different genes. Expression of each gene is directly used as an input of a clustering algorithm, which occupies an extremely large quantity of computing resources and includes a lot of useless information (not all genes are meaningful for cell clustering, for example, gene expression Housekeeping existing in all the cells is not meaningful for clustering). In addition, for the spatial transcriptomic data, spatial location information is a key to differentiate from single-cell data because single-cell transcriptomic data has only gene expression of a cell and does not know an absolute or relative location of the cell, while the spatial transcriptomic data is data that includes both the gene expression of the cell and knows the location of the cell. Therefore, for clustering analysis of the spatial transcriptomic data, how to effectively encode spatial location information into a feature is important content of a feature extraction process.

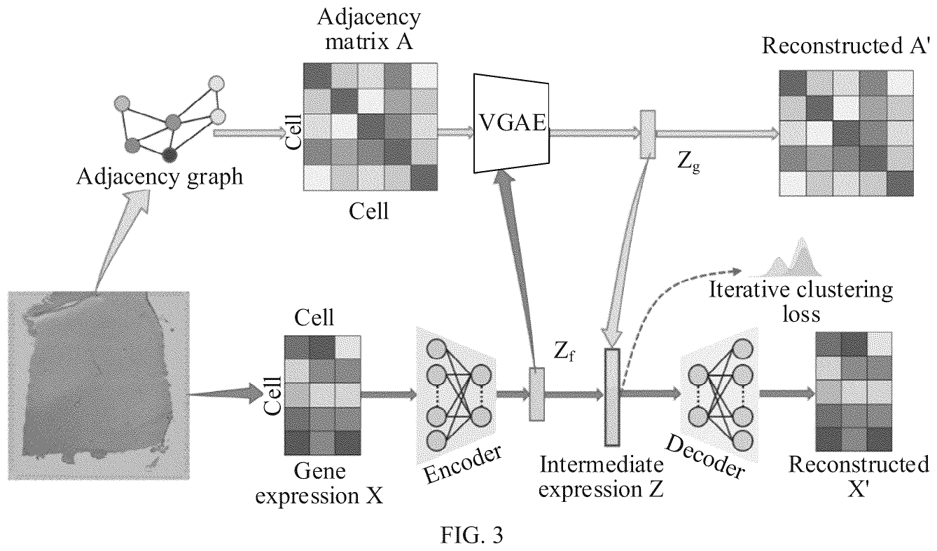

Currently, the spatial location information is encoded into feature information by using a scan equalization digital radiography (SEDR) method. Specifically, as shown in FIG. 3, spatial transcriptomic data is first parsed to obtain a domain matrix and a gene expression matrix, then the domain matrix is encoded by using a variational graph auto-encoder (VGAE), to obtain a feature Z_{g} that may represent spatial information, and the gene expression matrix is encoded by using an encoder, to obtain a feature Z_{f} that may represent gene information. The feature Z_{g} that may represent the spatial information is merged into the feature Z_{f} that may represent the gene information, to obtain intermediate expression Z. The intermediate expression Z includes both the spatial information and the gene information, and finally a subsequent task such as clustering can be implemented by using the intermediate expression Z.

It can be learned from FIG. 3 that a neighborhood relationship between cells is mainly used when the spatial information is encoded by using the SEDR, and if too few neighborhoods are considered when each cell is encoded, spatial location information cannot be effectively used. Therefore, to ensure effectiveness, a relatively large quantity of neighborhoods are considered, but considering the relatively large quantity of neighborhoods exponentially increases occupied computing resources, and the entire data processing process is relatively complex and has low efficiency.

To resolve the foregoing technical problems, in the embodiments of this application, target data is first obtained, and the target data is parsed to obtain spatial location information and first feature information of each object in N objects. The target data is converted into a first image based on the spatial location information and the first feature information of each object. Second feature information of each object in the N objects is extracted from the first image, the second feature information combining the spatial location information and the first feature information. In this way, preset processing (for example, object clustering or object annotation) is performed by using the second feature information of the N objects, to obtain an accurate processing result. That is, in the embodiments of this application, the target data is converted into the first image, the first image including the spatial location information and the first feature information of each object in the N objects, then feature extraction is performed on the first image, to obtain the second feature information of each object in the N objects, to encode the spatial location information into a feature, and preset processing such as clustering is directly performed on the second feature information including the spatial location information. The entire data processing process is simple, occupies less computing resources, and has high data processing efficiency. In addition, in the embodiments of this application, when the target data is processed, the target data is converted into the first image, and feature extraction is performed on the first image. When feature extraction is performed on the first image, only the second feature information of each object in the N objects is extracted. If the first image includes a pixel whose pixel value is zero, feature extraction is not performed on the pixel whose pixel value is zero in the first image, to further save the computing resources and improve the data processing efficiency.

The technical solution of the embodiments of this application is described in detail below through some embodiments. The following embodiments may be mutually combined, and same or similar concepts or processes may not be repeatedly described in some embodiments.

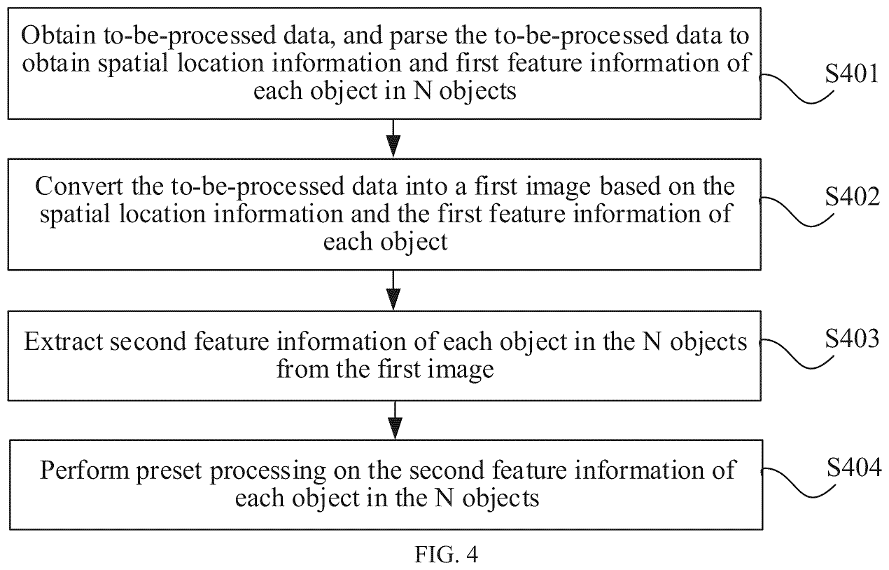

FIG. 4 is a schematic flowchart of a data processing method according to an embodiment of this application.

An execution body of this embodiment of this application may be an apparatus having a data processing function, for example, a data processing apparatus. In some embodiments, the data processing apparatus may be a server. In some embodiments, the data processing apparatus may be a terminal device. In some embodiments, the data processing apparatus may be a system formed by the server and the terminal device. Both the server and the terminal device may be understood as an electronic device. Therefore, for ease of description, an example in which the execution body is the electronic device is used for description.

As shown in FIG. 4, the data processing method in this embodiment of this application includes the following steps.

S401. Obtain target data, and parse the target data to obtain spatial location information and first feature information of each object in N objects.

N is a positive integer.

In some embodiments, N is a positive integer greater than 1, that is, there are a plurality of research objects in this embodiment of this application.

A specific type of the object is not limited in this embodiment of this application, for example, may be any object such as a cell, a sub-cell, or a cell cluster having spatial location information.

The target data may be understood as data corresponding to the N objects. The target data includes or implicitly includes the spatial location information and the first feature information of each object in the N objects.

In some embodiments, the target data may be generated by the electronic device.

In some embodiments, the target data may be sent by another device.

In some embodiments, the target data may alternatively be read by the electronic device from a storage device. For example, when receiving a processing request of the target data, the electronic device reads the target data from the storage device in response to the request.

A specific method for obtaining the target data is not limited in this embodiment of this application.

To accurately analyze and process the target data, in this embodiment of this application, after the target data is obtained, the target data is parsed, to obtain the spatial location information and the first feature information of each object in the N objects.

That is, in this embodiment of this application, each object in the N objects includes the spatial location information and the first feature information.

For example, each object in the N objects has different spatial location information.

The spatial location information may be understood as any information that may represent a spatial location of an object, for example, the spatial location information is location coordinates of a center point of the object.

For example, the first feature information of each object in the N objects may be the same or may be different, or may be partially the same or may be partially different. This is not limited in this embodiment of this application.

A specific type of the first feature information is not limited in this embodiment of this application.

In some embodiments, first feature information of an object may be understood as original feature information of the object included in the target data, and the original feature information may be understood as inherent feature (attribute) data of the object. For example, when the object is a cell, the first feature information may be understood as gene expression information of the cell.

The following describes a process of obtaining the spatial location information and the first feature information.

A specific implementation of parsing the target data to obtain the spatial location information and the first feature information of each object in the N objects in S401 is not limited.

In some embodiments, if the target data includes the spatial location information and the first feature information of each object in the N objects, the spatial location information and the first feature information of each object in the N objects may be directly read from the target data.

In some embodiments, at least one sub-object in this embodiment of this application may correspond to one object. For example, the object is a cell, and at least one sequencing point may be set on the cell. In this way, one sequencing point may be recorded as one sub-object corresponding to the cell.

In this embodiment, it is assumed that the target data includes first sub-feature information of a plurality of sub-objects, each sub-object in the plurality of sub-objects may correspond to one object in the N objects, and the first sub-feature information may be all or a part of the first feature information. Based on the foregoing, the plurality of sub-objects in the target data may correspond to the N objects. For example, a sub-object 11 and a sub-object 13 in the target data correspond to an object 1 in the N objects, and a sub-object 21 and a sub-object 22 in the target data correspond to an object 2 in the N objects. In this way, first sub-feature information of the sub-object 11 and the sub-object 13 may be used as first feature information of the object 1, and first sub-feature information of the sub-object 21 and the sub-object 22 may be used as first feature information of the object 2. Referring to the method, the first feature information of each object in the N objects may be determined.

In an implementation of this embodiment, the target data includes a correspondence between the plurality of sub-objects and the N objects. In this way, the electronic device may enable the plurality of sub-objects included in the target data to correspond to the N objects according to the correspondence between the plurality of sub-objects in the target data and the N objects, and use first sub-feature information corresponding to each object in the N objects as first feature information of the object.

In this embodiment, a method for determining the spatial location information of each object in the N objects includes at least the following examples.

Example 1: the target data includes the spatial location information of each object in the N objects. In this way, the electronic device may directly determine the spatial location information of each object in the N objects from the target data.

Example 2: the target data includes spatial location information of a sub-object. In this way, the electronic device may determine the spatial location information of the N objects according to spatial location information of a sub-object corresponding to each object in the N objects. For example, for an i^{th} object in the N objects, a sub-object corresponding to the i^{th} object is a sub-object i1 and a sub-object i2, and spatial location information of the i^{th} object may be determined according to spatial location information of the sub-object i1 and the sub-object i2. For example, an average value of the spatial location information of the sub-object i1 and the sub-object i2 is determined as the spatial location information of the i^{th} object.

A specific representation form of the spatial location information is not limited in this embodiment of this application.

In some embodiments, the spatial location information is represented in a form of a matrix.

For example, the spatial location information of the N objects is the following matrix A:

A={(x₁,y₁), (x₂, y₂), . . . (x₁,y₁), . . . (x_{N},y_{N})}

(xᵢ, yᵢ) represents the spatial location information of the i^{th} object in the N objects.

A specific representation form of the first feature information is not limited in this embodiment of this application.

In some embodiments, the first feature information is represented in a form of a matrix.

For example, the first feature information of the N objects is the following matrix B:

B={(a₁₁, a₁₂, . . . , a_{1G}), (a₂₁, a₂₂, . . . , a_{2G}), . . . , (aᵢ₁, aᵢ₂, . . . , a_{iG}), . . . , (a_{N1}, a_{N2}, . . . , a_{NG})}

(aᵢ₁, aᵢ₂, . . . , a_{iG}) represents G pieces of first feature information of the i^{th} object in the N objects. At least one piece of first feature information in the G pieces of first feature information of the i^{th} object may be empty. That is, G may be understood as total types of the first feature information included in the N objects, and types of the first feature information included in various objects in the N objects may be the same or may be different. That is, some objects in the N objects include different types of first feature information in the G pieces of first feature information.

In this embodiment of this application, after obtaining the target data according to the method, and parsing the target data to obtain the spatial location information and the first feature information of each object in the N objects, the electronic device performs the following S402.

S402. Convert the target data into a first image based on the spatial location information and the first feature information of each object.

In this embodiment of this application, the target data is converted into the first image, the first image including the spatial location information and the first feature information of each object in the N objects. Therefore, feature extraction is performed on the first image, to obtain second feature information of the N objects, the second feature information fusing the spatial location information and the first feature information. When subsequent data processing is performed based on the second feature information, a data processing effect can be improved.

In addition, in this embodiment of this application, the first feature information and the spatial location information are fused into the first image, and feature extraction is directly performed on the first image in which the spatial location information and the first feature information are fused, to encode the spatial location information into a feature. Compared with a manner of extracting a domain matrix, encoding the domain matrix, to obtain spatial encoding information, and fusing the spatial encoding information into a feature in SEDR, in this embodiment of this application, the entire data processing is simple, and occupies less computing resources. Further, in this embodiment of this application, the spatial location information of the N objects is directly used as spatial information, which can represent spatial information of an object more accurately than a domain relationship of the object. Further, data processing accuracy is improved, a data volume of the spatial information in this application is relatively small, and the occupied computing resources can be further reduced.

That is, in this embodiment of this application, the generated first image fuses the spatial location information and the first feature information of each object in the N objects. For example, each object in the N objects is used as a pixel whose pixel value is not zero in the first image, and the first feature information of the object is used as a channel of the first image, to form the first image.

Specifically, each object corresponds to a pixel in the first image (in this case, the first image is a blank image or an initial image) based on the spatial location information of each object in the N objects, and the first feature information of each object is used as a channel of the pixel corresponding to each object, so that the target data is converted into the first image, the converted first image including the spatial location information and the first feature information of each object in the target data.

A specific process of converting the target data into the first image in S402 is not limited in this embodiment of this application.

In some embodiments, each object in the N objects is used as a pixel in the first image according to the spatial location information of each object in the N objects, and the first feature information is used as a channel of the first image, to obtain the first image including N pixels.

In an example, the first image does not include a pixel whose pixel value is zero.

For example, N is 1000, and the first image includes 1000 pixels. Each pixel represents each object in 1000 objects, and sorting of the 1000 objects in the first image is determined according to spatial location information of the 1000 objects. It is assumed that spatial location information of each object is two-dimensional coordinates, for example, (x, y). The 1000 objects are arranged on a two-dimensional plane according to the spatial location information of each object, and there is no gap among the 1000 objects. Each object in the sorted 1000 objects is used as a pixel in the first image, to obtain the first image including 1000 pixels. In this embodiment, a method for sorting the 1000 objects according to the spatial location information is not limited. For example, the 1000 objects are first sorted according to sizes of x coordinates of the 1000 objects. If the x coordinates are the same, objects whose x coordinates are the same are sorted according to sizes of y coordinates, so that the 1000 objects are arranged on the two-dimensional plane.

In this embodiment, the obtained first image may be a rectangle or may not be a rectangle. This is not limited in this embodiment of this application.

In this embodiment, the first image does not include an invalid pixel whose pixel value is zero. Therefore, when feature extraction is performed on the first image, all valid information is extracted, so that reliability of feature extraction is improved, and computation is not performed on the invalid pixel whose pixel value is zero, thereby saving the computing resources.

In some embodiments, the target data may alternatively be converted into the first image through the following steps S402-A and S402-B.

S402-A. Create a blank second image.

S402-B. Fill the N objects into corresponding locations of the second image according to the spatial location information of each object in the N objects, and obtain the first image by using the first feature information as a channel of the first image.

In this embodiment, a blank second image is first created, that is, all pixels in the second image are pixels whose pixel values are zero. Then, the N objects are filled into corresponding locations in the blank second image one by one according to the spatial location information of each object in the N objects. For example, for an i^{th} object in the N objects, the i^{th} object is filled into a location corresponding to spatial location information of the i^{th} object in the second image, and so on, and the N objects may be filled into the blank second image. Then the first image may be obtained by using the first feature information as a channel of the first image.

A specific size of the second image is not limited in this embodiment of this application provided that it is ensured that the second image includes at least N pixels.

In some embodiments, if a quantity of pixels included in the second image is greater than N, the first image generated according to the second image includes N pixels whose pixel values are not zero and at least one pixel whose pixel value is zero, the N pixels whose pixel values are not zero being in one-to-one correspondence with the N objects.

A specific shape of the second image is not limited in this embodiment of this application.

In some embodiments, the second image may be a rectangle.

In some embodiments, the second image may be a regular shape except the rectangle, for example, a circle, an ellipse, or a polygon, or may be an irregular shape.

The following describes a process of creating the blank second image in S402-A.

A specific manner of creating the blank second image is not limited in this embodiment of this application.

In some embodiments, a size and a shape of the second image are preset.

In some embodiments, the second image is a rectangle or square image including at least N pixels.

In some embodiments, S402-A includes the following steps.

S402-A1. Create the blank second image according to the spatial location information of each object in the N objects.

In this embodiment, the blank second image is created according to the spatial location information of each object in the N objects, so that each object in the N objects may correspond to a pixel in the second image.

In this embodiment, the N objects may be relatively sparse, that is, a distance between objects in the N objects is relatively large, for example, at least a units. Therefore, if the spatial location information is not processed, a minimum distance between pixels whose pixel values are not zero in the formed first image is a pixels.

In a possible implementation of S402-A1, to reduce a size of the first image, when the blank second image is created, the spatial location information of the N objects may be reduced by a times, and the second image is created according to the spatial location information that is reduced by a times. For example, a maximum difference between x and a maximum difference between y of the spatial location information that is reduced by a times of the N objects are obtained, and the second image whose length is H and width is W is created by using the maximum difference between x as the length H of the second image and the maximum difference between y as the width W of the second image, a quantity of channels of the second image being total types G of the first feature information of the N objects. In this implementation, a size of the created blank second image is less than a size of a minimum enclosing rectangle of the N objects. Therefore, when the N objects are filled into the second image, to form the first image, a quantity of pixels whose pixel values are zero included in the formed first image is relatively small.

That there are a units between the N objects is only an example, and the distance depends on an actual condition.

In a possible implementation of S402-A1, S402-A1 includes the following steps.

S402-A11. Determine a minimum enclosing rectangle of the N objects according to the spatial location information of each object in the N objects.

S402-A12. Create the blank second image by using a size of the minimum enclosing rectangle as a size of the second image.

In this implementation, the blank second image is directly created according to the spatial location information of each object in the N objects without scaling the spatial location information, thereby simplifying the process of creating the second image and ensuring that the N objects can be filled into the blank second image without overlapping.

Specifically, a minimum value and a maximum value of coordinates of the N objects in an x-axis direction are determined, and a difference between the minimum value and the maximum value in the x-axis direction is determined as a length H of the minimum enclosing rectangle. Similarly, a minimum value and a maximum value of coordinates of the N objects in a y-axis direction are determined, and a difference between the minimum value and the maximum value in the y-axis direction is determined as a width W of the minimum enclosing rectangle, so that the minimum enclosing rectangle of the N objects is determined as a rectangle whose length is H and width is W. Then, the blank second image whose length is H and width is W is created by using a size of the minimum enclosing rectangle of the N objects as a size of the second image.

In this implementation, the size of the created second image is consistent with the size of the minimum enclosing rectangle of the N objects. In this way, it can be ensured that each object in the N objects corresponds to a pixel in the second image. Then, the N objects are filled into the blank second image one by one according to the spatial location information of each object in the N objects. For example, for an i^{th} object in the N objects, it may be determined that the i^{th} object corresponds to a jt pixel in the second image according to spatial location information of the i^{th} object, so that the i^{th} object may be filled into the j^{th} pixel in the second image, and so on, and the N objects may be filled into the blank second image according to the spatial location information of each object in the N objects to generate the first image.

For example, it is assumed that the spatial location information of the N objects is an N*2-dimensional matrix A, which is recorded as center coordinates of each object in the N objects. It is assumed that the first feature information of the N objects is an N*G-dimensional matrix B, which is recorded as G types of first feature information of each object in the N objects. For example, an i^{th} row and a j^{th} column of the matrix B represent an amount of j^{th} first feature information of an i^{th} object. Referring to the foregoing embodiments, the blank second image is first created according to the spatial location information of each object in the N objects, that is, the spatial location matrix B. The second image may be understood as a matrix of H*W*G, G representing a quantity of channels. Then, the N objects are filled into the second image according to the spatial location information of each object in the N objects to obtain the first image. For example, a value of a channel g in an h^{th} row and a w^{th} column, that is, a pixel of (h, w, g) in the first image represents an amount of a g^{th} first feature information of an object whose center coordinates are (h, w).

According to the method, in the generated first image, each object in the N objects occupies a pixel, and the pixel occupied by the object is recorded as a pixel whose pixel value is not zero. All channels of the pixel whose pixel value is not zero form all first feature information of the object. A pixel whose pixel value is zero in the first image represents that there is no object at the location, and values of all channels of the pixel whose pixel value is zero are 0. Therefore, in the generated first image of HAW, only the N pixels whose pixel values are not zero include information, and another pixel whose pixel value is zero does not include information.

For example, it is assumed that the generated first image is represented by using a matrix C as follows:

$$C = {[ \begin{matrix}▯ & ⊗ & … & ▯ \\ ⊗ & ▯ & … & ⊗ \\ … & … & … & … \\ ▯ & ▯ & … & ⊗\end{matrix} ]}_{H \times W}$$

□ represents a pixel whose pixel value is zero, and ⊗ represents a pixel whose pixel value is not zero, that is, a pixel in which an object is located.

In this embodiment of this application, after the target data is converted into the first image according to the step, the following step S403 is performed.

S403. Extract second feature information of each object in the N objects from the first image.

It can be learned from the foregoing that in this embodiment of this application, the target data is converted into the first image based on the spatial location information and the first feature information of the N objects, the first image including the spatial location information and the first feature information of each object in the N objects. Then, feature extraction is performed on the first image, to obtain the second feature information of each object in the N objects, so that the generated second feature information fuses the spatial location information and the first feature information of the N objects, and when subsequent data processing is performed by using the second feature information, data processing accuracy can be improved.

That is, in this embodiment of this application, the second feature information may be understood as a feature vector obtained by performing feature extraction on the first image, the feature vector fusing spatial location information and first feature information of an object.

A specific implementation of extracting the second feature information of each object in the N objects from the first image in S403 is not limited in this embodiment of this application.

In some embodiments, it can be learned from the foregoing that if the first image includes N pixels whose pixel values are not zero and does not include a pixel whose pixel value is zero, feature extraction may be performed on the first image by using any feature extraction method, to obtain the second feature information of each object in the N objects. Because the first image does not include the pixel whose pixel value is zero, there is no waste of computing resources caused by processing an invalid pixel whose pixel value is zero, thereby saving the computing resources.

In some embodiments, it can be learned from the foregoing that in addition to including the N pixels whose pixel values are not zero, the first image further includes at least one pixel whose pixel value is zero, for example, as shown in the matrix C. In this embodiment, to save the computing resources, the electronic device performs feature extraction on only the pixel whose pixel value is not zero in the first image, and does not perform feature extraction on the pixel whose pixel value is zero in the first image, to obtain the second feature information of each object in the N objects.

A specific implementation of performing feature extraction on the pixel whose pixel value is not zero in the first image and skipping performing feature extraction on the pixel whose pixel value is zero in the first image is not limited in this embodiment of this application.

In a possible implementation of this embodiment, S403 includes the following steps S403-A and S403-B.

S403-A. Perform feature extraction on the first image, to obtain a first feature map.

The first feature map includes N non-zero elements in one-to-one correspondence with the N objects.

In this implementation, feature extraction is performed on the first image, to obtain a first feature map, the first feature map including N non-zero elements, and the N non-zero elements being in one-to-one correspondence with the N objects, that is, the N non-zero elements of the first feature map being in one-to-one correspondence with the N pixels whose pixel values are not zero in the first image.

In this step, a size of the obtained first feature map may be the same as or may be different from a size of the first image. This is not limited in this embodiment of this application.

In some embodiments, the size of the first feature map obtained in this step is the same as the size of the first image, a location of a zero element in the first feature map is consistent with a location of a pixel whose pixel value is zero in the first image, and a location of a non-zero element in the first feature map is consistent with a location of a pixel whose pixel value is not zero in the first image.

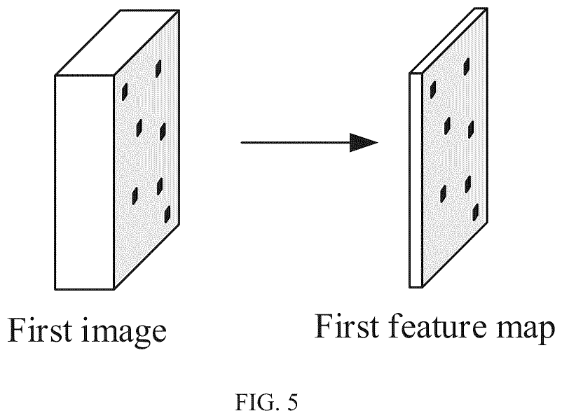

For example, as shown in FIG. 5, a left side in FIG. 5 is a first image, a black square in the first image represents an object or a pixel whose pixel value is not zero, and there are a total of seven objects. A right side in FIG. 5 is a first feature map, a size of the first feature map is consistent with a size of the first image, a black square in the first feature map represents a non-zero element, and there are a total of seven non-zero elements. locations of the seven non-zero elements are consistent with locations of the seven objects in the first image, and a quantity and locations of zero elements in the first feature map are also consistent with a quantity and locations of pixels whose pixel values are zero in the first image. It can be learned from FIG. 5 that in this embodiment of this application, when feature extraction is performed on the first image, to generate the first feature map, feature extraction is performed on only pixels whose pixel values are not zero (that is, objects) in the first image, and feature extraction is not performed on a pixel whose pixel value is zero in the first image, thereby avoiding processing invalid information, further saving the computing resources, and improving data processing efficiency.

A specific manner of extracting the first feature map from the first image in S403-A is not limited in this embodiment of this application.

In some embodiments, feature extraction is performed on the first image by using a submanifold sparse convolution-based autoencoder, to obtain the first feature map.

Submanifold sparse convolution (SubMConv) is also referred to as a valid sparse convolution, and feature extraction on the pixel whose pixel value is zero in the first image may be skipped.

The following compares the submanifold sparse convolution with ordinary convolution and ordinary sparse convolution.

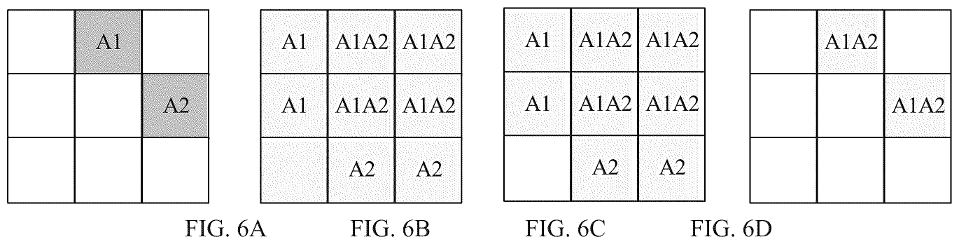

FIG. 6A to FIG. 6D show a difference among ordinary convolution, ordinary sparse convolution, and submanifold sparse convolution. FIG. 6A is an input sparse image. As shown in FIG. 6A, the sparse image includes two pixels A1 and A2 whose pixel values are not zero, and others are all pixels whose pixel values are zero. FIG. 6B to FIG. 6D are respectively outputs after the input image shown in FIG. 6A is processed through ordinary convolution, ordinary sparse convolution, and submanifold sparse convolution when a size of a convolution kernel is 3. In FIG. 6B to FIG. 6D, blank light gray cells indicate no information, cells with text indicate that information is included, and white cells indicate that no storage is required at the locations.

When a convolution operation is performed by using a convolution kernel with a size of 3, which is equivalent to performing convolution on each cell, information of the cell and a total of at most nine cells around the cell is processed. FIG. 6A is used as an example. When convolution is performed on a cell at the bottom left corner, there is no effective information that needs to be processed. Therefore, a value of 0 is returned. In ordinary convolution, the value of zero needs to be stored. Therefore, information at the location needs to be used as an input when a next round of convolution is performed. In ordinary sparse convolution, the value of zero does not participate in the operation. Therefore, the location does not need to be stored when the value of 0 is returned. In submanifold sparse convolution, the value of zero does not participate in the operation, and only content at a location corresponding to a non-zero value in an original input is saved. Therefore, after the submanifold sparse convolution is performed, sizes of an output and an input are completely consistent with a non-zero value location and a zero value location. It can be learned that the use of the submanifold sparse convolution greatly reduces content that participates in computation and needs to be stored, thereby greatly reducing computing performance requirements and occupation of computing resources.

A quantity of submanifold sparse convolution layers included in a submanifold sparse convolution-based autoencoder is not limited in this embodiment of this application. For example, there is at least one submanifold sparse convolution layer.

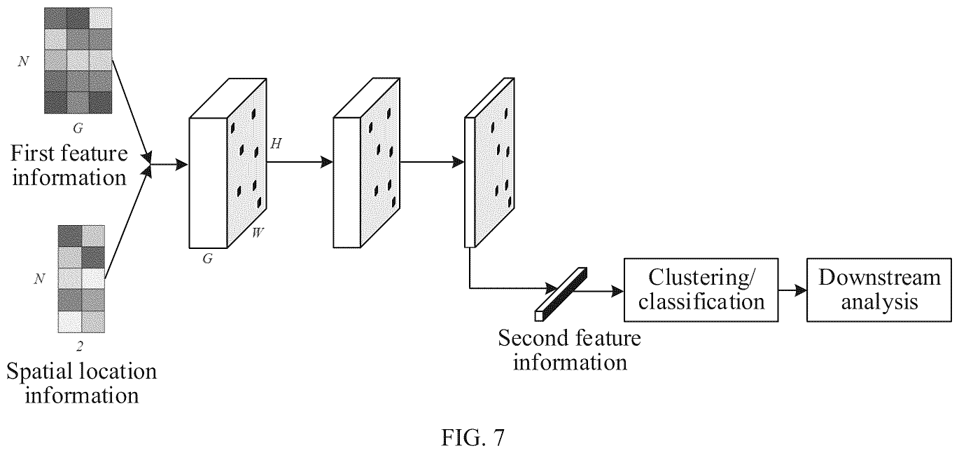

In some embodiments, the submanifold sparse convolution-based autoencoder includes two submanifold sparse convolution layers. In this case, the data processing process provided in this embodiment of this application may be represented as that shown in FIG. 7.

Specifically, as shown in FIG. 7, target data is obtained, and the target data is parsed to obtain spatial location information and first feature information of each object in N objects, the spatial location information of the N objects being an N*2-dimensional matrix, and the first feature information of the N objects being an N*G-dimensional matrix. Then, the target data is converted into a first image based on the spatial location information and the first feature information of each object. For example, as shown in FIG. 7, a size of the first image is H*W, and a quantity of channels is G. The first image is inputted into the submanifold sparse convolution-based autoencoder, and is compressed through a first submanifold sparse convolution layer in a channel direction, to obtain a feature map of H*W*R. After the feature map of H*W*R is compressed through a second submanifold sparse convolution layer, a first feature map of H*W*L is generated. That is, in this embodiment of this application, for encoding layers in the submanifold sparse convolution-based autoencoder, the first image is compressed by using two submanifold sparse convolution layers in a direction of a quantity of channels, and after two submanifold sparse convolutions are performed, an input X with a size of H*W*G is encoded into a first feature map with a size of H*W*L.

In some embodiments, in this embodiment of this application, the submanifold sparse convolution-based autoencoder may include a decoding layer, the decoding layer including at least one submanifold sparse deconvolution layer.

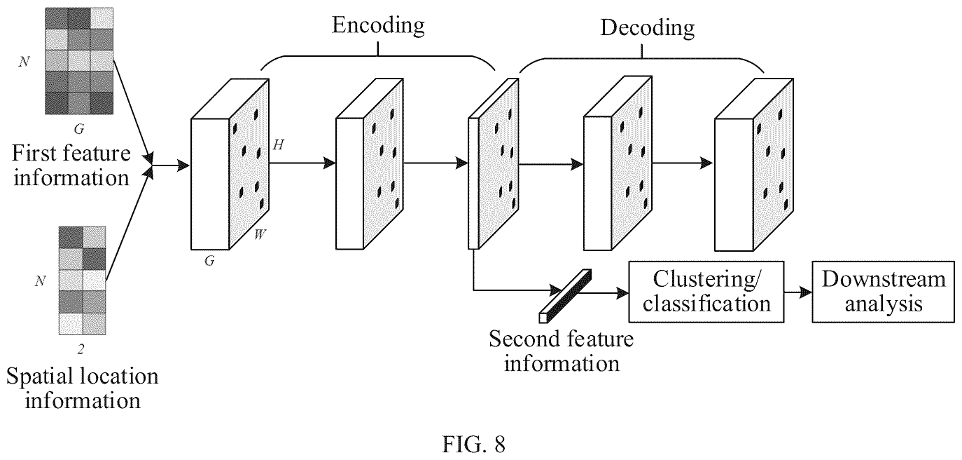

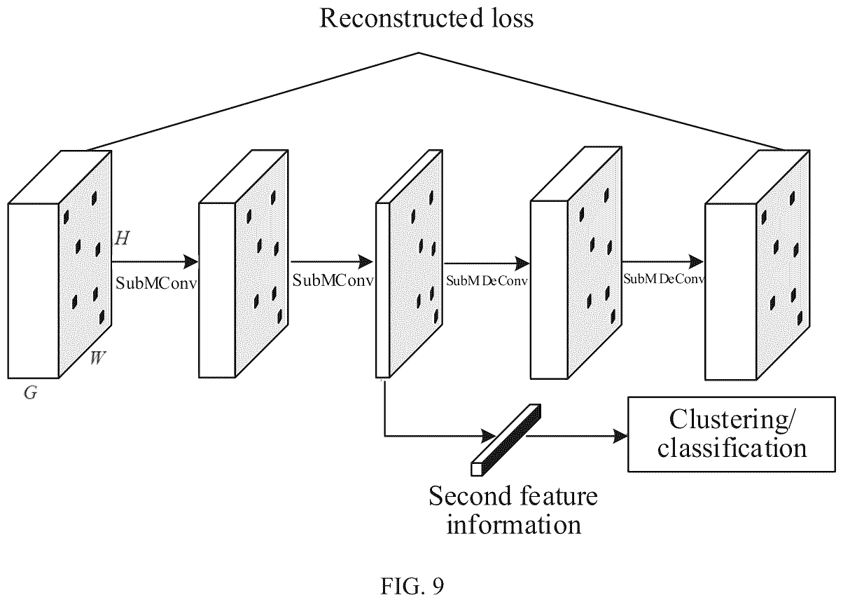

In an example, as shown in FIG. 8 and FIG. 9, the submanifold sparse convolution-based autoencoder includes an encoding layer and a decoding layer, the encoding layer including two submanifold sparse convolution (SubMConv) layers, and the decoding layer including two submanifold sparse deconvolution (SubDeMConv) layers. When the submanifold sparse convolution-based autoencoder is trained, the encoding layer compresses a training image by using the two submanifold sparse convolution layers in a channel direction, to encode an input X with a size of H*W*G into a feature map with a size of H*W*L. Then, the decoding layer decodes the feature map with the size of H*W*L again by using the two submanifold sparse deconvolution layers to a reconstructed matrix X′ with a size of H*W*G. Then, a reconstructed loss between X and X′ is optimized, to train the submanifold sparse convolution-based autoencoder.

In some embodiments, the submanifold sparse convolution-based autoencoder may use a relatively large convolution kernel, to ensure that information about a specific quantity of surrounding objects can be considered during each sparse convolution.

In some embodiments, the submanifold sparse convolution-based autoencoder may include only the encoding layer and does not include the decoding layer. In this case, the encoding layer may be trained by introducing a label or another guidance.

During actual use, feature extraction is performed on the first image by using the encoding layer in the submanifold sparse convolution-based autoencoder, to obtain a first feature map.

According to the method, after feature extraction is performed on the first image, to obtain the first feature map, the following step S403-B is performed.

S403-B. Obtain the second feature information of each object in the N objects according to feature information of the N non-zero elements in the first feature map.

It can be learned from the foregoing that the generated first feature map includes feature information of the N non-zero elements in one-to-one correspondence with the N objects, so that the second feature information of each object in the N objects may be obtained according to the feature information of the N non-zero elements in the first feature map.

For example, the first feature map is determined as the second feature information of the N objects.

In another example, feature information of non-zero elements is extracted from the first feature map, and the feature information of the non-zero elements is determined as the second feature information of the N objects. For example, for an i^{th} object in the N objects, the i^{th} object corresponds to a j^{th} non-zero element in the first feature map. Therefore, feature information of the j^{th} non-zero element is determined as second feature information of the i^{th} object. For example, as shown in FIG. 7, only N non-zero elements are valid in the generated first feature map with the size of H*W*L. After all zero elements are removed, an N*L-dimensional feature may be finally obtained, to form an input representation Embedding of a subsequent task for subsequent analysis.

According to the method, after feature extraction is performed on the first image, to obtain the second feature information of each object in the N objects, the following step S404 is performed

S404. Perform preset processing on the second feature information of each object in the N objects.

The preset processing may be understood as a preset downstream task performed based on the second feature information.

A specific manner of preset processing is not limited in this embodiment of this application.

In some embodiments, the preset processing includes at least one of performing clustering on the N objects, performing annotation on the N objects, and downstream analysis.

In an example, if the preset processing is performing clustering on the N objects, S404 includes the following steps.

S404-A. Perform clustering on the N objects according to the second feature information of each object in the N objects, to obtain M cluster sets of the N objects.

Each cluster set in the M cluster sets includes at least one object, M being a positive integer less than or equal to N.

It can be learned from the foregoing that in this embodiment of this application, the second feature information fuses the spatial location information and the first feature information, and when clustering is performed based on the second feature information, clustering accuracy can be improved.

A specifically used clustering algorithm is not limited in this embodiment of this application, for example, may be a general clustering algorithm such as K-means or KNN, or may be a clustering algorithm such as Louvain or Leiden based on community detection.

In some embodiments, this embodiment of this application further includes: selecting P cluster sets from the M cluster sets, P being a positive integer less than or equal to M; and performing clustering on objects in the P cluster sets again. For example, the objects in the P cluster sets are merged, or the objects in the P cluster sets are classified into more detailed categories, to further implement multi-level and multi-step clustering.

In an example, if the preset processing is to perform annotation on the N objects, in this embodiment of this application, a type annotation model may be provided. The type annotation model includes the submanifold sparse convolution-based autoencoder and a classifier, so that end-to-end type annotation of an object can be implemented. For example, a probability value that an object that is obtained from another marked data or obtained manually may belong to a type may be used as a counterfeiting label, the counterfeiting label is connected after the extracted second feature information through a classifier, and a loss of the classifier is optimized when the reconstructed loss is optimized, so that a type annotation model that can directly implement end to end can be finally obtained, and performance of the type annotation model is far better than that obtained by directly using the counterfeiting label as an annotation result.

In some embodiments, related downstream analysis may be further performed on the target data according to a result of the clustering or the type annotation. This is not limited in this embodiment of this application.

According to the data processing method provided in this embodiment of this application, target data is obtained, and the target data is parsed to obtain spatial location information and first feature information of each object in N objects. The target data is converted into a first image based on the spatial location information and the first feature information of each object. Second feature information of each object in the N objects is extracted from the first image, the second feature information combining the spatial location information and the first feature information. In this way, preset processing (for example, object clustering or object annotation) is performed by using the second feature information of the N objects, to obtain an accurate processing result. That is, in the embodiments of this application, the target data is converted into the first image, the first image including the spatial location information and the first feature information of each object in the N objects, then feature extraction is performed on the first image, to obtain the second feature information of each object in the N objects, to encode the spatial location information into a feature, and preset processing such as clustering is directly performed on the second feature information including the spatial location information. The entire data processing process is simple, occupies less computing resources, and has high data processing efficiency. In addition, in the embodiments of this application, when the target data is processed, the target data is converted into the first image, and feature extraction is performed on the first image. When feature extraction is performed on the first image, only the second feature information of each object in the N objects is extracted. If the first image includes a pixel whose pixel value is zero, feature extraction is not performed on the pixel whose pixel value is zero in the first image, to further save the computing resources and improve the data processing efficiency.

The following further describes a data processing process provided in this embodiment of this application by using an example in which the object is a cell, the target data is spatial transcriptomic data, and the first feature information is gene information.

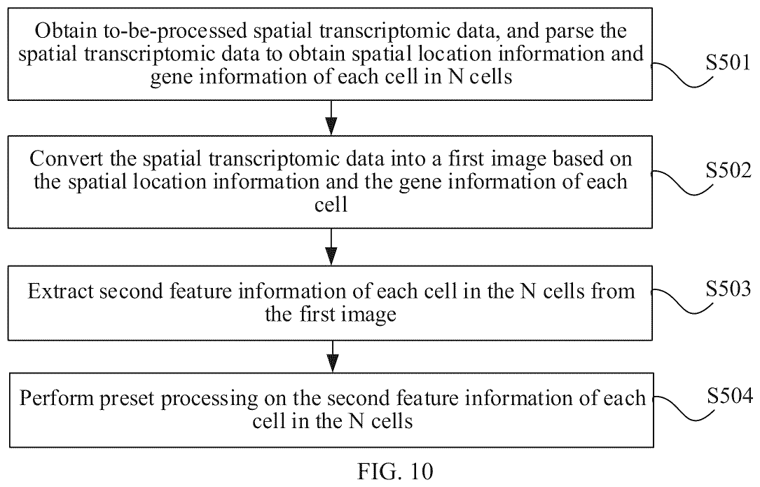

FIG. 10 is a schematic diagram of a processing process of spatial transcriptomic data according to an embodiment of this application. As shown in FIG. 10, this embodiment of this application includes the following steps.

S501. Obtain to-be-processed spatial transcriptomic data, and parse the spatial transcriptomic data to obtain spatial location information and gene information of each cell in N cells.

In some embodiments, the spatial transcriptomic data includes the spatial location information and the first feature information of each cell in the N cells. In this case, the spatial location information and the first feature information of each cell in the N cells may be directly read from the spatial transcriptomic data.

In some embodiments, the spatial transcriptomic data includes gene expression captured by a sequencing point. In this case, S501 includes the following steps.

S501-A. Enable sequencing points in the spatial transcriptomic data to correspond to the N cells, to obtain the gene information of each cell in the N cells, the gene information of the cell including gene expression captured by a sequencing point corresponding to the cell.

During actual test, at least one sequencing point may be set on the cell to capture gene expression on the sequencing point, to form spatial transcriptomic data, the spatial transcriptomic data including the gene expression captured by the sequencing point. In this embodiment of this application, after the spatial transcriptomic data is obtained, the spatial transcriptomic data is parsed to obtain the spatial location information and the gene information of each cell in the N cells. Specifically, various sequencing points in the spatial transcriptomic data correspond to the N cells according to a mapping relationship between cells and sequencing points, and gene expression captured by the sequencing points corresponding to the cells forms the gene information of the cells, to encapsulate original sequencing data in a form of a sequencing point into gene expression data in a form of a cell.

In an example, the spatial transcriptomic data includes the spatial location information of each cell in the N cells, and then the spatial location information of each cell in the N cells may be directly read from the spatial transcriptomic data.

In another example, the spatial transcriptomic data includes spatial location information of each sequencing point, then spatial location information of a sequencing point corresponding to a cell is determined according to a mapping relationship between the sequencing point and the cell, and spatial location information of the cell is determined.

In some embodiments, the sequencing points in the spatial transcriptomic data correspond to the N cells. Before the gene information of each cell in the N cells is obtained, the method further includes: removing an incorrect sequencing point in the spatial transcriptomic data, and then enabling the sequencing points after the incorrect sequencing point is removed in the spatial transcriptomic data to correspond to the N cells, to obtain the gene information of each cell in the N cells.

In some embodiments, the gene information of the N cells generated according to the method is an N*G-dimensional matrix, N being a quantity of cells, G being a quantity of gene types, and an i^{th} row and a j^{th} column of the matrix representing a quantity of genes j expressed by a cell i.

In some embodiments, the spatial location information of the N cells generated according to the method is an N*2-dimensional matrix, which is recorded as center coordinates of each cell.

S502. Convert the spatial transcriptomic data into the first image based on the spatial location information and the gene information of each cell.

A specific implementation process of S502 is consistent with that of S402. For details, reference may be made to the detailed descriptions of S402.

Specifically, each cell corresponds to a pixel in the first image (in this case, the first image is a blank image or an initial image) based on the spatial location information of each cell and the gene information of each cell is used as a channel of the pixel corresponding to each cell, so that the spatial transcriptomic data is converted into the first image, the converted first image including the spatial location information and the gene information of each cell during conversion of the spatial transcriptomic data.

In some embodiments, a blank second image is created; and the N cells are filled into corresponding locations of the second image according to the spatial location information of each cell in the N cells, and the first image is obtained by using the gene information as a channel of the first image.

A method for creating the blank second image may include: creating the blank second image according to the spatial location information of each cell in the N cells.

For example, a minimum enclosing rectangle of the N cells is determined according to the spatial location information of each cell in the N cells. The blank second image is created by using a size of the minimum enclosing rectangle as a size of the second image.

In some embodiments, the first image includes N pixels whose pixel values are not zero and that are in one-to-one correspondence with the N cells, and a quantity of channels of the first image is a quantity of gene types of the N cells.

It is assumed that the first image is an image of H*W*G, H*W being a size of the first image, and G being a quantity of channels of the first image, a value of a channel g (that is a pixel (h, w, g)) in an h^{th} row and a w^{th} column in the first image represents a quantity of genes g expressed by a cell whose center coordinates are (h, w).

When the generated first image includes a pixel whose pixel value is not zero and a pixel whose pixel value is zero, each cell occupies the pixel whose pixel value is not zero, all channels of the pixel whose pixel value is not zero form gene expression of the cell, it indicates that there is no cell at the location, and values of all channels of the pixel whose pixel value is zero is 0.

Only N pixels in all H*W pixels of the generated first image include information.

In some embodiments, in this embodiment of this application, only information about the pixel whose pixel value is not zero in the first image is extracted. Therefore, when the first image is generated, it is not necessary to reduce the pixel whose pixel value is zero included in the first image through a required size of the first image, to compress the resource occupation, but conversion may be directly performed according to the spatial location information of the N cells to obtain the first image.

S503. Extract second feature information of each cell in the N cells from the first image.

A specific implementation process of S503 is consistent with that of S403. For details, reference may be made to detailed descriptions of S403.

In some embodiments, feature extraction is performed on the first image, to obtain a first feature map, and second feature information of each cell in the N cells is obtained according to feature information of N non-zero elements in the first feature map.

In an example, a size of the first feature map is the same as a size of the first image, a location of a zero element in the first feature map is consistent with a location of a pixel whose pixel value is zero in the first image, and a location of a non-zero element in the first feature map is consistent with a location of a pixel whose pixel value is not zero in the first image.

In some embodiments, feature extraction is performed on the first image by using a submanifold sparse convolution-based autoencoder, to obtain the first feature map.

In some embodiments, the submanifold sparse convolution-based autoencoder includes at least one submanifold sparse convolution layer.

For a specific implementation of S503, reference is made to the descriptions of S403. Details are not described herein again.

S504. Perform preset processing on the second feature information of each cell in the N cells.

For example, clustering, or annotation, or downstream analysis, or the like is performed on the N cells according to the second feature information of each cell in the N cells.

In some embodiments, clustering is performed on the N cells according to the second feature information of each cell in the N cells, to obtain M cluster sets of the N cells, each cluster set in the M cluster sets including at least one cell, and M being a positive integer less than or equal to N.

In some embodiments, after the M cluster sets are obtained, P cluster sets may be further selected from the M cluster sets, P being a positive integer less than or equal to M; and clustering is performed on cells in the P cluster sets again.

In some embodiments, this embodiment of this application is applicable to a single-cell analysis platform and is used as a part of an analysis process of the spatial transcriptomic data. A user inputs spatial transcriptomic data (that is, gene expression and corresponding spatial locations of a series of cells). The spatial transcriptomic data is first converted into a first image, then second feature information of each cell is extracted by using a submanifold sparse convolution-based autoencoder, and all the cells are divided into a plurality of different clusters by using a clustering algorithm. Clustering may be directly applied to all data or may be applied to partial data according to user selection from a plurality of levels (for example, ⅓ data is first selected and classified into eight classes, and then an eighth class is selected and classified into 10 small classes). Clustering having a low level and a low quantity of clusters may assist in analysis of an anatomical structure, and clustering having a high level and a high quantity of clusters may assist in analysis of a cell or sub-cell structure type. Downstream analysis such as gene spatial differential expression, a Marker gene, and a highly variable gene (HVG) may also be performed according to a result of the clustering.

It can be learned from the foregoing that the data processing method provided in the embodiments of this application can reduce occupation of computing resources and improve data processing efficiency. Therefore, the data processing method provided in this embodiment of this application is applicable to spatial transcriptomic data with an ultra-high resolution and a large field of view. The resolution refers to a sequencing point density, and low-resolution data refers to that the sequencing point density is greater than a cell density. In this case, a sequencing point represents a sum of gene expression of a plurality of cells. A high resolution or ultra-high resolution refers to that the sequencing point density is equal to or greater than a cell density. In this case, a single sequencing point represents a cell or sub-cell structure, and one or more sequencing points are merged to form gene expression of one cell. The field of view refers to a size of a total space occupied by all cells in a batch of data, the spatial transcriptomic data is usually obtained through sequencing on an issue slice, and the large field of view refers to that the slice is relatively large, and generally means that there are a relatively large quantity of cells.

To further describe an effect of the data processing method provided in this embodiment of this application, the beneficial effects of this embodiment of this application are described through experiments.

Table 1 shows GPU view memory occupations of this application and an SEDR technology in different data amounts.

TABLE 1
Video memory occupations of this application
and an SEDR technology

| | Data amount | GPU video memory | |
|---|---|---|---|
| Method | (quantity of cells) | occupations | |
| SEDR | 24510 | 22252 | MB |
| This application | 24510 | 1403 | MB |
| This application | 73530 | 3073 | MB |

It can be learned from Table 1 that the video memory occupation in this application is significantly relatively low and this application is efficiently applicable to the spatial transcriptomic data with the ultra-high resolution and the large field of view.

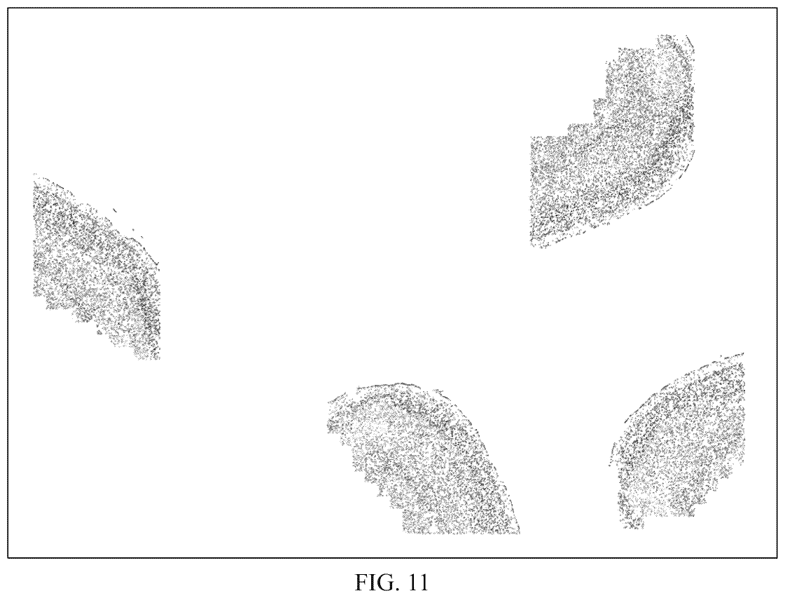

In addition, when cell type guidance is introduced, end-to-end annotation of a cell type can be directly implemented in this application. Generally, there is no relatively reliable verification index for a clustering result, and it is difficult to perform effective quantitative comparison. Therefore, directly comparing an annotation result can more conveniently reflect the superiority of this application. FIG. 11 is a schematic diagram of an annotation result (which corresponds to a single cell as type guidance) on one sample in rat brain primary motor cortex data in multiplexed error-robust fluorescence in situ hybridization (MERFISH) according to this application. Table 2 shows accuracy of cell type annotation results generated by using corresponding single cell data as type guidance on a mouse 1_sample1 sample of the data set through this application and other six comparison automatic annotation methods (Seurat, SingleR, scmap, Cell-ID, scNym, and SciBet).

TABLE 2
Accuracy of this application and comparison methods

| | This | | | | | | |
|---|---|---|---|---|---|---|---|
| | application | Seurat | SingleR | scmap | Cell-ID | scNym | SciBet |
| Accuracy | 90.13% | 85.50% | 87.68% | 64.54% | 16.45% | 78.69% | 88.06% |

It can be learned from Table 2 that annotation accuracy of this application is higher than annotation accuracy of other annotation methods.

In addition, because gene expression and spatial information are effectively combined in this application as self-supervision for training, it is also excellent in performance of noisy data. Table 3 shows accuracy of this application and other six comparison methods when 30% genes are removed manually on a mouse 1_sample1 sample of MERFISH data.

TABLE 3
Accuracy of this application and comparison methods

| | This | | | | | | |
|---|---|---|---|---|---|---|---|
| | application | Seurat | SingleR | scmap | CellID | scNym | SciBet |
| Accuracy | 85.02% | 81.36% | 80.15% | 53.51% | 15.41% | 78.66% | 81.37% |

It can be learned from Table 3 that annotation accuracy of the method provided in this embodiment of this application is significantly higher than that of other methods after noise is added to data.

According to the data processing method provided in this embodiment, to-be-processed spatial transcriptomic data is obtained, the spatial transcriptomic data is parsed to obtain spatial location information and gene information of each cell in N cells. The spatial transcriptomic data is converted into a first image based on the spatial location information and the gene information of each cell, and then second feature information of each cell in the N cells is extracted from the first image, the second feature information fusing the spatial location information and the gene information, so that when preset processing is performed by using the second feature information of each cell in the N cells, an accurate processing result may be obtained. That is, in this application, the spatial transcriptomic data is converted into the first image, the first image including the spatial location information and the gene information of each cell in the N cells, and then feature extraction is performed on the first image, to obtain the second feature information of each cell in the N cells, to encode the spatial location information into a feature. The entire data processing process is simple, occupies less computing resources, and has high data processing efficiency. In addition, in this embodiment of this application, when feature extraction is performed on the first image, only the second feature information of the N cells is extracted. When the first image includes a pixel whose pixel value is zero, feature extraction is not performed on the pixel whose pixel value is zero in the first image, to further save the computing resources and improve the data processing efficiency.

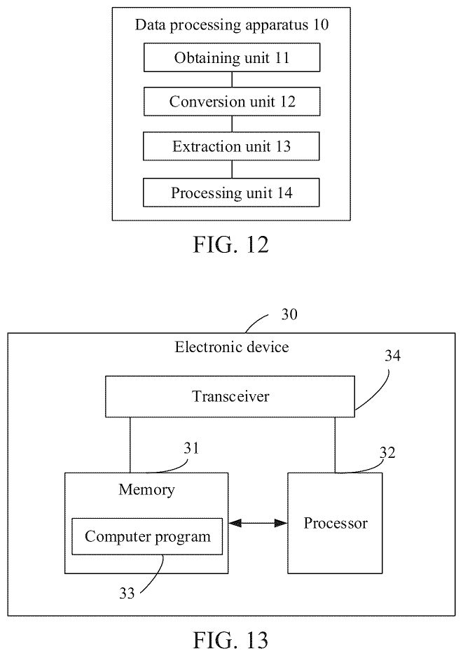

The method embodiments of this application are described in detail above with reference to FIG. 4 to FIG. 11, and apparatus embodiments of this application are described below with reference to FIG. 12 and FIG. 13.

FIG. 12 is a schematic block diagram of a data processing apparatus according to an embodiment of this application. The apparatus 10 may be an electronic device or may be a part of an electronic device.

As shown in FIG. 12, the data processing apparatus 10 includes:
an obtaining unit 11, configured to obtain target data, and parse the target data to obtain spatial location information and first feature information of each object in N objects, N being a positive integer;
a conversion unit 12, configured to convert the target data into a first image based on the spatial location information and the first feature information of each object;
an extraction unit 13, configured to extract second feature information of each object in the N objects first image; and
a processing unit 14, configured to perform preset processing on the second feature information of each object in the N objects.

In some embodiments, the conversion unit 12 is further configured to create a blank second image; and fill the N objects into corresponding locations of the second image according to the spatial location information of each object in the N objects, and obtain the first image by using the first feature information as a channel of the first image.

In some embodiments, the conversion unit 12 is further configured to create the blank second image according to the spatial location information of each object in the N objects.

In some embodiments, the conversion unit 12 is further configured to determine a minimum enclosing rectangle of the N objects according to the spatial location information of each object in the N objects; and create the blank second image by using a size of the minimum enclosing rectangle as a size of the second image.

In some embodiments, the first image includes N pixels whose pixel values are not zero and at least one pixel whose pixel value is zero, the N pixels whose pixel values are not zero being in one-to-one correspondence with the N objects.

In some embodiments, the extraction unit 13 is further configured to perform feature extraction on the first image, to obtain a first feature map, the first feature map including N non-zero elements in one-to-one correspondence with the N objects; and obtain the second feature information of each object in the N objects according to feature information of the N non-zero elements in the first feature map.

In some embodiments, a size of the first feature map is the same as a size of the first image, a location of a zero element in the first feature map is consistent with a location of a pixel whose pixel value is zero in the first image, and a location of a non-zero element in the first feature map is consistent with a location of a pixel whose pixel value is not zero in the first image.

In some embodiments, the extraction unit 13 is further configured to perform feature extraction on the first image by using a submanifold sparse convolution-based autoencoder, to obtain the first feature map.

In some embodiments, the submanifold sparse convolution-based autoencoder includes at least one submanifold sparse convolution layer.

In some embodiments, the preset processing includes at least one of performing clustering on the N objects, performing annotation on the N objects, and downstream analysis.

In some embodiments, when the preset processing is performing clustering on the N objects the processing unit 14 is further configured to perform clustering on the N objects according to the second feature information of each object in the N objects, to obtain M cluster sets of the N objects, each cluster set in the M cluster sets including at least one object, and M being a positive integer less than or equal to N.

In some embodiments, the processing unit 14 is further configured to select P cluster sets from the M cluster sets, P being a positive integer less than or equal to M; and perform clustering on objects in the P cluster sets again.

In some embodiments, the object is a cell, the target data is spatial transcriptomic data, and the first feature information is gene information, and
the obtaining unit 11 is further configured to obtain to-be-processed spatial transcriptomic data, and parse the spatial transcriptomic data to obtain spatial location information and gene information of each cell in N cells;
the conversion unit 12 is further configured to convert the spatial transcriptomic data into the first image based on the spatial location information and the gene information of each cell;
the extraction unit 13 is further configured to extract second feature information of each cell in the N cells from the first image; and
the processing unit 14 is further configured to perform preset processing on the second feature information of each cell in the N cells.

In some embodiments, the first image includes N pixels whose pixel values are not zero and that are in one-to-one correspondence with the N cells, and a quantity of channels of the first image is a quantity of gene types of the N cells.

In some embodiments, the spatial transcriptomic data includes gene expression captured by a sequencing point, and the obtaining unit 11 is further configured to enable sequencing points in the spatial transcriptomic data to correspond to the N cells, to obtain the gene information of each cell in the N cells, the gene information of the cell including gene expression captured by a sequencing point corresponding to the cell.

In some embodiments, the obtaining unit 11 is further configured to remove an incorrect sequencing point in the spatial transcriptomic data; and enable the sequencing points after the incorrect sequencing point is removed in the spatial transcriptomic data to correspond to the N cells, to obtain the gene information of each cell in the N cells.

It is to be understood that the apparatus embodiment may correspond to the method embodiment, and for similar description, reference may be made to the method embodiment. To avoid repetition, details are not described herein again. Specifically, the apparatus shown in FIG. 12 may execute the data processing method embodiments, and the foregoing and other operations and/or functions of modules in the apparatus are separately configured for implementing the method embodiments. For brevity, details are not described herein.

The apparatus in this embodiment of this application is described in the foregoing from the perspective of a functional module with reference to the accompanying drawings. It is to be understood that the functional module may be implemented in a form of hardware, or may be implemented through an instruction in a form of software, or may be implemented in a combination of a hardware module and a software module. Specifically, steps in the foregoing method embodiments in the embodiments of this application may be completed by using a hardware integrated logical circuit in the processor, or by using instructions in a form of software. Steps of the methods disclosed with reference to the embodiments of this application may be directly performed and completed by using a hardware decoding processor, or may be performed and completed by using a combination of hardware and a software module in the decoding processor. In some embodiments, the software module may be located in a mature storage medium in the field, such as a random access memory, a flash memory, a read-only memory, a programmable read-only memory, an electrically-erasable programmable memory, or a register. The storage medium is located in the memory. The processor reads information in the memory and completes the steps of the foregoing method embodiments in combination with hardware thereof.

FIG. 13 is a schematic block diagram of an electronic device according to an embodiment of this application. The electronic device is configured to perform the data processing method embodiment.

As shown in FIG. 13, the electronic device 30 may include:
a memory 31 and a processor 32. The memory 31 is configured to store a computer program 33 and transmit the computer program 33 to the processor 32. The processor 32 may invoke the computer program 33 from the memory 31 and run the computer program 33, to implement the method in the embodiments of this application.

For example, the processor 32 may be configured to perform the foregoing method step executed by instructions in the computer program 33.

In some embodiments, the processor 32 may include, but not limited to,
a general-purpose processor, a digital signal processor (DSP), an application-specific integrated circuit (ASIC), a field programmable gate array (FPGA), or another programmable logical device, discrete gate or transistor logical device, or discrete hardware component, and the like

In some embodiments of this application, the memory 31 includes, but not limited to,
a volatile memory and/or a non-volatile memory. The non-volatile memory may be a read-only memory (ROM), a programmable ROM (PROM), an erasable programmable read-only memory (EPROM), an electrically EPROM (EEPROM), or a flash memory. The volatile memory may be a random access memory (RAM), and is used as an external cache. Through exemplary but not limitative description, many forms of RAMs may be used, for example, a static random access memory (SRAM), a dynamic random access memory (DRAM), a synchronous dynamic random access memory (SDRAM), a double data rate synchronous dynamic random access memory (DDR SDRAM), an enhanced synchronous dynamic random access memory (ESDRAM), a synchlink dynamic random access memory (SLDRAM) and a direct rambus random access memory (DR RAM).

In some embodiments of this application, the computer program 33 may be divided into one or more modules. The one or more modules are stored in the memory 31 and executed by the processor 32, to complete the data processing method provided in this application. The one or more modules may be a series of computer program instruction sections that can complete a specific function, and the instruction section is configured for describing an execution process of the computer program 33 in the electronic device.

As shown in FIG. 13, the electronic device 30 may further include:
a transceiver 34, where the transceiver 34 may be connected to the processor 32 or the memory 31.

The processor 32 may control the transceiver 34 to communicate with another device, specifically, may send information or data to the another device, or receive information or data sent by the another device. The transceiver 34 may include a transmitter and a receiver. The transceiver 34 may further include an antenna, and a quantity of the antenna can be one or more.

It is to be understood that various components of the electronic device 30 are connected to each other by a bus system. In addition to including a data bus, the bus system further includes a power bus, a control bus, and a status signal bus.

According to an aspect of this application, a computer storage medium is provided. The computer storage medium stores a computer program. When the computer program is executed by a computer, the computer is enabled to perform the method in the foregoing method embodiments.

An embodiment of this application further provides a computer program product including instructions. When the instructions are executed by a computer, the computer is enabled to perform the method in the foregoing method embodiments.

According to another aspect of this application, a computer program product or a computer program is provided, the computer program product or the computer program including computer instructions, the computer instructions being stored in a computer-readable storage medium. The processor of the computer device reads the computer instructions from the computer-readable storage medium, and the processor executes the computer instructions, to cause the electronic device to perform the method in the method embodiments.

In other words, when software is used to implement embodiments, all or a part of embodiments may be implemented in a form of a computer program product. The computer program product includes one or more computer instructions. When the computer program instructions are loaded and executed on the computer, the procedures or functions according to the embodiments of this application are all or partially generated. The computer may be a general-purpose computer, a dedicated computer, a computer network, or other programmable apparatuses. The computer instructions may be stored in a computer-readable storage medium or may be transmitted from a computer-readable storage medium to another computer-readable storage medium. For example, the computer instructions may be transmitted from a website, computer, server, or data center to another website, computer, server, or data center in a wired (for example, a coaxial cable, an optical fiber, or a digital subscriber line (DSL)) or wireless (for example, infrared, radio, or microwave) manner The computer-readable storage medium may be any available medium that a computer can access or a data storage device such as a server or a data center that includes one or more integrated available media. The available medium may be a magnetic medium (such as a floppy disk, a hard disk, or a magnetic tape), an optical medium (such as a digital video disc (DVD)), a semiconductor medium (such as a solid state disk (SSD)) or the like.

A person of ordinary skill in the art may be aware that, in combination with the examples described in the embodiments disclosed in this specification, modules and algorithm steps may be implemented by electronic hardware or a combination of computer software and electronic hardware. Whether the functions are performed by hardware or software depends on particular applications and design constraint conditions of the technical solutions. A person skilled in the art may use different methods to implement the described functions for each particular application, but it is to not be considered that the implementation goes beyond the scope of this application.

In the several embodiments provided in this application, it can be understood that the disclosed system, apparatus, and method may be implemented in other manners. For example, the described apparatus embodiment is merely exemplary. For example, the module division is merely logical function division and may be other division in actual implementation. For example, a plurality of modules or components may be combined or integrated into another system, or some features may be ignored or not performed. In addition, the displayed or discussed mutual couplings or direct couplings or communication connections may be implemented by using some interfaces. The indirect couplings or communication connections between the apparatuses or modules may be implemented in electronic, mechanical, or other forms.

The modules described as separate parts may or may not be physically separate, and parts displayed as modules may or may not be physical modules, may be located in one position, or may be distributed on a plurality of network units. Some or all of modules may be selected to achieve the objective of the embodiment solutions according to an actual need. For example, functional modules in the embodiments of this application may be integrated into one processing module, or each of the modules may exist alone physically, or two or more modules are integrated into one module.

In this application, the term “unit” or “module” refers to a computer program or part of the computer program that has a predefined function and works together with other related parts to achieve a predefined goal and may be all or partially implemented by using software, hardware (e.g., processing circuitry and/or memory configured to perform the predefined functions), or a combination thereof. Each unit or module can be implemented using one or more processors (or processors and memory). Likewise, a processor (or processors and memory) can be used to implement one or more modules or units. Moreover, each module or unit can be part of an overall module that includes the functionalities of the module or unit. The foregoing content is merely specific implementations of this application, but are not intended to limit the protection scope of this application. Any variation or replacement readily figured out by a person skilled in the art within the technical scope disclosed in this application shall fall within the protection scope of this application. Therefore, the protection scope of this application shall be subject to the protection scope of the claims.

What is claimed is:

1. A data processing method, comprising:
obtaining target data;
parsing the target data to obtain spatial location information and first feature information of each object in the target data;
converting the target data into a first image based on the spatial location information and the first feature information of each object in the target data;
extracting second feature information of each object in the target data from the first image; and
performing preset processing on the second feature information of each object in the target data.

2. The method according to claim 1, wherein the converting the target data into a first image based on the spatial location information and the first feature information of each object in the target data comprises:
creating a blank second image;
filling each object in the target data into a corresponding location of the second image according to the spatial location information of the object; and
obtaining the first image by using the first feature information as a channel of the first image.

3. The method according to claim 2, wherein the creating a blank second image comprises:
creating the blank second image according to the spatial location information of each object in the target data.

4. The method according to claim 2, wherein the creating the blank second image according to the spatial location information of each object in the target data comprises:
determining a minimum enclosing rectangle of the objects in the target data according to the spatial location information of the objects; and
creating the blank second image by using a size of the minimum enclosing rectangle as a size of the second image.

5. The method according to claim 1, wherein the first image comprises N pixels whose pixel values are not zero and at least one pixel whose pixel value is zero, the N pixels whose pixel values are not zero being in one-to-one correspondence with the N objects.

6. The method according to claim 1, wherein the extracting second feature information of each object in the target data from the first image comprises:
performing feature extraction on the first image, to obtain a first feature map, the first feature map comprising N non-zero elements in one-to-one correspondence with the objects; and
obtaining the second feature information of each object in the objects according to feature information of the N non-zero elements in the first feature map.

7. The method according to claim 1, wherein the performing preset processing on the second feature information of each object in the target data comprises:
performing clustering on the objects according to the second feature information of each object in the target data, to obtain multiple cluster sets of the objects, each cluster set comprising at least one object in the target data.

8. An electronic device, comprising a processor and a memory,
the memory being configured to store a computer program; and
the processor being configured to execute the computer program, to implement a data processing method including:
obtaining target data;
parsing the target data to obtain spatial location information and first feature information of each object in the target data;
converting the target data into a first image based on the spatial location information and the first feature information of each object in the target data;
extracting second feature information of each object in the target data from the first image; and
performing preset processing on the second feature information of each object in the target data.

9. The electronic device according to claim 8, wherein the converting the target data into a first image based on the spatial location information and the first feature information of each object in the target data comprises:
creating a blank second image;
filling each object in the target data into a corresponding location of the second image according to the spatial location information of the object; and
obtaining the first image by using the first feature information as a channel of the first image.

10. The electronic device according to claim 9, wherein the creating a blank second image comprises:
creating the blank second image according to the spatial location information of each object in the target data.

11. The electronic device according to claim 9, wherein the creating the blank second image according to the spatial location information of each object in the target data comprises:
determining a minimum enclosing rectangle of the objects in the target data according to the spatial location information of the objects; and
creating the blank second image by using a size of the minimum enclosing rectangle as a size of the second image.

12. The electronic device according to claim 8, wherein the first image comprises N pixels whose pixel values are not zero and at least one pixel whose pixel value is zero, the N pixels whose pixel values are not zero being in one-to-one correspondence with the N objects.

13. The electronic device according to claim 8, wherein the extracting second feature information of each object in the target data from the first image comprises:
performing feature extraction on the first image, to obtain a first feature map, the first feature map comprising N non-zero elements in one-to-one correspondence with the objects; and
obtaining the second feature information of each object in the objects according to feature information of the N non-zero elements in the first feature map.

14. The electronic device according to claim 8, wherein the performing preset processing on the second feature information of each object in the target data comprises:
performing clustering on the objects according to the second feature information of each object in the target data, to obtain multiple cluster sets of the objects, each cluster set comprising at least one object in the target data.

15. A non-transitory computer-readable storage medium, configured to store a computer program, the computer program, when executed by a processor of an electronic device, causing the electronic device to perform a data processing method including:
obtaining target data;
parsing the target data to obtain spatial location information and first feature information of each object in the target data;
converting the target data into a first image based on the spatial location information and the first feature information of each object in the target data;
extracting second feature information of each object in the target data from the first image; and
performing preset processing on the second feature information of each object in the target data.

16. The non-transitory computer-readable storage medium according to claim 15, wherein the converting the target data into a first image based on the spatial location information and the first feature information of each object in the target data comprises:
creating a blank second image;
filling each object in the target data into a corresponding location of the second image according to the spatial location information of the object; and
obtaining the first image by using the first feature information as a channel of the first image.

17. The non-transitory computer-readable storage medium according to claim 16, wherein the creating a blank second image comprises:
creating the blank second image according to the spatial location information of each object in the target data.

18. The non-transitory computer-readable storage medium according to claim 15, wherein the first image comprises N pixels whose pixel values are not zero and at least one pixel whose pixel value is zero, the N pixels whose pixel values are not zero being in one-to-one correspondence with the N objects.

19. The non-transitory computer-readable storage medium according to claim 15, wherein the extracting second feature information of each object in the target data from the first image comprises:
performing feature extraction on the first image, to obtain a first feature map, the first feature map comprising N non-zero elements in one-to-one correspondence with the objects; and
obtaining the second feature information of each object in the objects according to feature information of the N non-zero elements in the first feature map.

20. The non-transitory computer-readable storage medium according to claim 15, wherein the performing preset processing on the second feature information of each object in the target data comprises:
performing clustering on the objects according to the second feature information of each object in the target data, to obtain multiple cluster sets of the objects, each cluster set comprising at least one object in the target data.
